(19) United States
(12) Patent Application Publication
(10) Pub. No.: US 2025/0196419 A1
(43) Pub. Date: Jun. 19, 2025

(54) THERMOPLASTIC FILM AND PACKAGING MADE OF SUCH THERMOPLASTIC FILM USEFUL TO STERILIZE OBJECTS

(71) Applicant: VISCOFAN ESPAÑA, S.L.U., TAJONAR (ES)

(72) Inventor: LORENZO LIZCANO DE VEGA, NOÁIN (ES)

(21) Appl. No.: 18/848,809

(22) PCT Filed: Mar. 21, 2023
(86) PCT No.: PCT/EP2023/057217

§ 371 Date: Sep. 19, 2024

(30) Foreign Application Priority Data
Mar. 22, 2022 (EP) 22382266.9

(51) Int. Cl. B29C 48/08; A61L 2/26; B29C 48/00; B29C 48/21; B29C 71/02; B29K 77/00; B29K 105/00; B29L 31/00; B65D 65/40

(52) U.S. Cl. CPC B29C 48/08; A61L 2/26; B29C 48/022; B29C 48/21; B29C 71/02; B65D 65/40; B29C 2071/022; B29K 2039/06; B29K 2077/00; B29K 2105/0038; B29K 2105/0067; B29K 2105/0088; B29K 2995/0012; B29K 2995/0067; B29L 2031/712

(57) ABSTRACT

A thermoplastic film including: a) a polyamide in an amount in the range of from 60-85% by weight; b) not cross-linked PVP in an amount in the range of from 13 to 25% by weight; and c) a plasticizer in an amount in the range of from 1 to 4% by weight; and a specific additive, wherein the amounts of each of the components are with respect to the total amount of components of the film. A packaging including one or more such single multilayer thermoplastic films joined by at least one seal seam, or adhesive bonded. The use of the packaging to sterilize objects or materials, in particular, by means of water vapor or ethylene oxide.

This application claims the benefit of European Patent Application EP22382266 filed 22 Mar. 2022.

### TECHNICAL FIELD

The invention relates to the field of polymeric films and sterilizable packaging such as bags or pouches made of these films. It also relates to their preparation process and their uses to sterilize objects or materials by means of water vapor or ethylene oxide.

### BACKGROUND ART

Many healthcare products are stored in sealed sterilization packaging until its use. Sterilization packaging for medical material typically consists of special sterilizable paper and plastic foils or laminates. The paper is a water strength paper having a certain porosity, and the plastic laminates generally is a polyester/polypropylene laminate such as PETP/PP and both form the packaging. However, the mechanical strength of the paper packaging is poor, especially after the vapor sterilization and it has the drawback that pulp fiber from the packaging is easy detached contaminating the packed object.

There have been several attempts to modify the packaging by replacing the paper foils by non-woven materials. For instance, CA1314265C discloses a disposable sterilization packaging especially for packing hospitals utensils. It comprises partly of a plastic material and partly of a non-woven or other material. Thus, part is a laminate of a non-paper non-woven inner layer made of fibers of polypropylene, polyester, polyamide or polyethylene or any combination of the four, and of an outer perforated layer made of a plastic sheet material.

US20200407142A1 discloses a packaging for the sterile storage and transport of objects for medical, pharmaceutical, or cosmetic applications. The packaging includes at least one bag made of a first and a second web, bonded to one another, the first web consists of a selectively permeable nonwoven fabric and the second web consists of a laminated film with at least three layers, where the first layer is a polymer film arranged on the external side of the bag such as a polyester or polyamide, the second layer is a lamination adhesive, and the third layer is a polymer film such as an homopolymer or copolymer of polyethylene, propylene, or polyester, arranged on the internal side of the bag.

WO2006/012482 discloses an autoclavable sterilization bag having first and second composite film laminates positioned in opposing relation and sealed to one another to form a pouch. At least the first laminate comprises a heat sealable transparent thermoplastic polymer inner layer such as a polyolefin film forming an inner surface of the laminate, a transparent polymer film outer layer such as of polyethylene terephthalate, nylon (polyamide), polypropylene, polyethylene, and cellophane, and a transparent barrier layer located between the inner and outer layers and formed of molecularly oriented polychlorotrifluoroethylene (PCTFE). According to this document, the use of molecularly oriented PCTFE films provide a structure with sufficient barrier properties to replace metal foil for use in ethylene oxide gamma sterilization, e-beam sterilization, and autoclave sterilization.

Commercial microfiber nonwoven fabrics to cover tubes and trays to be used for sterilization packaging made of high density polyethylene are also known. For instance, Tyvek is a commercial non-woven fabric which is selectively permeable allowing to use ethylene oxide or steam to sterilize the space within the tube or the tray.

One of the drawbacks of the known packaging's is the difficulty to ensure and impermeable and durable seal seam. In case of undersealing the layers are not sufficiently bonded and therefore, exhibits low adhesion. In case of over sealing small holes termed pinholes in the seal seam can appear that can be caused by excessive temperatures, pressures or sealing time. Materials such as Tyvek can also suffer from these problems because the defects are sometimes not discernible during conventional inspection. Another drawback is that they may suffer from heat shrinkage. If the packaging shrinks (e.g., after autoclave treatment), the appearance of the packaging becomes significantly worse, and it becomes difficult for the user to handle and open the packaging. Besides, as in the case of using paper, non-woven materials are not transparent and for some applications, a transparent packaging is desirable.

On the other hand, blends of polyamide with polyvinylpyrrolidone have been used for preparing polymeric films in a very different field, specifically, in the field of food products, having properties appropriate for such field. For instance, EP1380212B1 describes a heat-shrinkable sausage casing film, which is made of a blend of polyamide and cross-linked polymer of N-vinylpyrrolidone, with moderate water vapor permeability and moderate oxygen impermeability but allows the passage of smoke products and thus allows the sausage to be smoked. Also, WO02078455A1 describes a single-layer polymeric film for food products, comprising a polyamide matrix and a component providing high permeability in respect to smoke substances and/or water vapors. This component is a hydrophilic compound which is present in an amount of 4.5-50.0 wt. %, forming in the polyamide base matrix a highly dispersed phase with a linear domain size of 0.1-3.0 μm in a direction perpendicular to a plane of the film. The hydrophilic compound can be homopolymers and/or copolymers such as vinylpyrrolidone or polyvinyl alcohol among other polymers.

From what is known in the art, it is derived that there is still the need to find polymeric films, having low porosity, that do not suffer from significant heat shrinkage, and with good mechanical properties to be used for packaging in the medical, cosmetical, and pharmaceutical fields in order to ensure the sterility of the contents.

### SUMMARY OF INVENTION

The inventors have developed a thermoplastic film comprising a polyamide, non-crosslinked polyvinyl pyrrolidone (PVP), and a plasticizer in specific amounts, and which is flexible, monolithic, high water vapor permeable, low oxygen permeable, and has excellent mechanical properties. These properties make these thermoplastic films appropriate for the purposes of the present invention. The inventors have also developed a packaging comprising single or multilayer thermoplastic films joined by at least one seal seam or adhesive bonded which are useful, among other things, for sterilization of objects or materials, in particular, with water vapor or ethylene oxide.

Mixtures of polyamides with polyvinyl pyrrolidone have been used in the food field for cases for smoked, dried, and/or fermented food products. However, as far as the inventors know the specific mixtures of the present invention, which differs from these mixtures mainly in that they comprise non-cross linked polyvinyl pyrrolidone and in the specific percentages of components, have not been previously suggested for the purposes of the present invention (sterilization of objects) and they have been found to have a set of properties that make them particularly suitable to be used to prepare films and packaging for such purposes.

Unlike paper or Tyvek that show porosity, the packaging of the present invention is a monolithic-non-porous-film which reduces the risk of microbial contamination from their sterilization until the use for the final user. The packaging according to the invention does not have fibers whereas paper films do. This fact eliminates the risk of contamination from fibers detached from the film when opening the sterilized packaging. A further advantage is that the entire packaging is transparent/translucent versus paper or Tyvek/plastic packaging. Furthermore, the film of the present invention presents excellent properties to be used for sterilization. They have a high puncture resistance; high surface tension of PVP which helps to prevent the formation of water droplets on the inner surface of the packaging, and high water permeability but lower than that of paper, thus the packaging allows autoclaving of a moisture-sensitive product and protects the sterilized product stored in the packaging from moisture better than paper. Besides, it shows dimensional stability (shrinkage≤2% or ≤1% or ≤0.5%). Finally, a further advantage is that the film of the present invention does not have odor.

Thus, an aspect of the present invention relates to a film comprising: a) a polyamide in an amount in the range of from 60-85% by weight, b) not cross-linked PVP in an amount in the range of from 13 to 25% by weight, and c) a plasticizer in an amount in the range of from 1 to 4% by weight, and further comprise an additive selected from the group consisting of a water repellent, a slipping agent, an additive for providing color, anti-blocking agent, a mineral filler, a nucleating agent, a UV block additive, a surfactant, and mixtures thereof; wherein the amounts of each of the components are with respect to the total amount of components of the film.

Another aspect of the present invention relates to a packaging comprising one or more single or multilayer thermoplastic films as defined above, joined by at least one seal seam or adhesive bonded.

Another aspect of the present invention relates to a process for preparing the film as defined above, comprising the following steps: a) melt blending the individual components that will form the film which are at least a polyamide in an amount in the range of from 60-85% by weight, not cross-linked PVP in an amount in the range of from 13 to 25% by weight, and a plasticizer in an amount in the range of from 1 to 4% by weight, and an additive selected from the group consisting of a water repellent, a slipping agent, an additive for providing color, anti-blocking agent, a mineral filler, a nucleating agent, a UV block additive, a surfactant, and mixtures thereof; wherein the amounts of each of the components are with respect to the total amount of components of the film; b) extruding the mixture by a process selected from a mono-orientation process, a bi-orientation process, a blown film not oriented extrusion process, and a cast film not oriented extrusion process; and c) in case of oriented film process, thermally anneal the film.

Another aspect of the present invention relates to a process for preparing the packaging as defined above, which comprises joining the film by a method selected from sealing, and adhesive bonding.

Another aspect of the present invention relates to the use of a packaging as defined above, to sterilize objects or materials, for instance, by means of water vapor or ethylene oxide.

### BRIEF DESCRIPTION OF DRAWINGS

FIG. 1A shows several samples before sterilization (Example 2, Example 3, and comparative Example 1A).

FIG. 1B shows several samples after sterilization (Example 2, Example 3, and comparative Example 1A).

FIG. 2 shows the maximum sealing strength for Example 2, Example 3, and comparative examples.

FIG. 3 shows the puncture strength (N) for Example 2, Example 3, and comparative examples.

FIG. 4A shows the tensile strength/elongation at break MD (Machine film direction) for Example 2, Example 3, and comparative examples.

FIG. 4B shows the tensile strength/elongation at break TD (Transversal film direction) for Example 2, Example 3, and comparative examples.

FIG. 5 shows the oxygen transmission rate for Example 2, Example 3, and comparative examples.

FIG. 6 shows the tearing strength MD/TD (N) for Example 2, Example 3, and comparative examples.

FIG. 7 shows the water vapor transmission rate (WVTR) for Example 2, Example 3, and comparative examples.

FIG. 8 shows the water vapor uptake for Example 2, Example 3, and comparative examples.

### DETAILED DESCRIPTION OF THE INVENTION

The term “packaging” as used herein refers to any type of container such as bags, pouches, etc. Bags requires that at least one of the sides has not been sealed, or adhesive-bonded. They can be produced from a tube or from a folded flat film. Pouches are defined herein as having a seal seam or adhesive bond at all three sides and are produced from two or more separate films. The closed pouch has a sealed bond, or adhesive bond at all sides.

The term “thermoplastic film” as used herein refers to a plastic polymer material that becomes pliable or mouldable at a certain elevated temperature and solidifies upon cooling.

The term “permeable” refers to a film with a water vapor transmission rate of at least 250 g/m² per day measured for the film before sterilization by the method detailed in the examples section.

As used herein, the term “monolithic” film may comprise any film that is continuous and substantially free or free of pores. In certain alternative embodiments of the invention, a “monolithic” film may comprise fewer pore structures than would otherwise be found in a microporous film. Monolithic films are continuous and free of pores. Monolithic breathable films are capable of absorbing gas and water molecules on one surface, transferring the molecules through the film, and releasing the molecules on the opposite surface.

The term “melting” refers to a transition stage between a fully crystalline or partially crystalline endothermic peak in the second heating of DSC curve. Melting temperatures exhibited in this invention correspond to Differential Scanning calorimetry test performed at heating and cooling rates 20° K/min with 20 minutes holding time at 573° K (299.9° C.) after first heating run and before first cooling run.

The term “cross-linking” refers to a bond or a short sequence of bonds that links one polymer chain to another. These links may take the form of covalent bonds or ionic bonds. The term “not cross-linked” when refers to PVP refers to the absence of cross-linking between different polymeric chains of PVP. Not cross-linked PVP has a linear structure and is soluble in water. Thus not-cross linked PVP is equivalent to soluble PVP being the solubility of PVP in water at 25° C.≥100 mg/ml.

It is part of the present invention a thermoplastic film comprising: a) a polyamide in an amount in the range of from 60-85% by weight, b) not cross-linked PVP in an amount in the range of from 13 to 25% by weight, and c) a plasticizer in an amount in the range of from 1 to 4% by weight, which further comprise an additive selected from the group consisting of a water repellent, a slipping agent, an additive for providing color, anti-blocking agent, a mineral filler, a nucleating agent, a UV block additive, a surfactant, and mixtures thereof; wherein the amounts of each of the components are with respect to the total amount of components of the film. The sum of the components adds up to 100 wt %.

The thermoplastic film of the present invention is a packaging thermoplastic film which is suitable for sterilization of objects or materials, for instance, by means of water vapor or ethylene oxide.

In a particular embodiment, the thermoplastic film comprises a) a polyamide in an amount in the range of from 60-85% by weight, b) not cross-linked PVP in an amount in the range of from 13 to 25% by weight, and c) a plasticizer in an amount in the range of from 1 to 4% by weight, wherein the thermoplastic film does not comprise a polyvinyl alcohol, and wherein the thermoplastic film further comprises an additive selected from the group consisting of a water repellent, a slipping agent, an additive for providing color, anti-blocking agent, a mineral filler, a nucleating agent, a UV block additive, a surfactant, and mixtures thereof; wherein the amounts of each of the components are with respect to the total amount of components of the thermoplastic film.

In another particular embodiment, the thermoplastic film comprises a) a polyamide in an amount in the range of from 60-85% by weight, b) not cross-linked PVP in an amount in the range of from 13 to 25% by weight, and c) a plasticizer in an amount in the range of from 1 to 4% by weight, wherein a), b), and c) are the only polymers of the thermoplastic film, and wherein the thermoplastic film further comprises an additive selected from the group consisting of a water repellent, a slipping agent, an additive for providing color, anti-blocking agent, a mineral filler, a nucleating agent, a UV block additive, a surfactant, and mixtures thereof; wherein the amounts of each of the components are with respect to the total amount of components of the thermoplastic film.

In another particular embodiment, the thermoplastic film consists of a) a polyamide in an amount in the range of from 60-85% by weight, b) not cross-linked PVP in an amount in the range of from 13 to 25% by weight, and c) a plasticizer in an amount in the range of from 1 to 4% by weight, d) an additive selected from the group consisting of a water repellent, a slipping agent, an additive for providing color, anti-blocking agent, a mineral filler, a nucleating agent, a UV block additive, a surfactant, and mixtures thereof; wherein the amounts of each of the components are with respect to the total amount of components of the thermoplastic film.

In another particular embodiment, the thermoplastic film is that from those mentioned above or below where there is at least an additive which is a water repellent selected from the group consisting of a N,N′—(C₁₆-C₂₂)-alkylenebis amide and triglyceride esters of fatty acids ranging from C₈-C₃₀. In a particular embodiment, the N,N′—(C₁₆-C₂₂)-alkylenebis amide is an N,N′-alkylene bisamide of a fatty acid. Examples of N,N′-alkylene bisamides of fatty acids are N,N′-ethylene-bis-stearamide, N,N′-ethylene-bis-oleamide, N,N′ethylene-bis-palmitamide, gadoleamide, erucamide, N,N′-dioleyl adipamide, N,N′-dierucylamide, and stearamides such as ethylene bis stearamide (EBS). EBS can also act as a slip agent, surface lubricant, and antistatic additive. In a particular embodiment, the thermoplastic film is that from those mentioned above or below where the water repellent is EBS. The presence of the water repellent contributes to prevent the formation of water droplets on the inner surface of the packaging and also provides good antiblocking properties.

In another particular embodiment, the thermoplastic film is that where the anti-blocking agent is selected from the group consisting of calcium carbonate, silica, and talc.

In another particular embodiment, the thermoplastic film is that where the additive for providing color is selected from the group consisting of: inorganic pigments, organic pigments, and colorants.

In another particular embodiment, the thermoplastic film is that which comprises, a mineral filler which is selected from the group consisting of quartz powder, titanium dioxide, calcium carbonate, talc, mica, aluminosilicates, glass stable fibers, mineral fibers, and microglass beads.

In another particular embodiment, the thermoplastic film is that where the slipping agent is selected from the group consisting of: N,N′-alkylenebisamide, an oleoamide, and a metal salt of stearic acid. Examples of metal salts are those where the metal is calcium, zinc, magnesium, or sodium.

In another particular embodiment, the thermoplastic film is that where the polyamide has a melting point below 200° C., in particular, the polyamides have a melting point between 130° C. and below 200° C. These polyamides facilitate the thermosealing and reduce the welding force of the thermoplastic film. This property is especially useful in the case of using such thermoplastic films for preparing a packaging for medical, pharmaceutical, or cosmetic materials since it has an influence in the heat sealing.

In another particular embodiment, the thermoplastic film is that where the polyamide is selected from the group consisting of aliphatic homopolyamide, aliphatic copolyamide, partially aromatic homopolyamide, partially aromatic Copolyamide, ter-polyamides, and mixtures thereof.

In another particular embodiment, the thermoplastic film is that where the polyamide is selected from the group consisting of a homopolyamide, and an aliphatic copolyamide obtainable by total or partial polymerization of ε-caprolactam and not crosslinked 2-polyvinyl pyrrolidone (PVP).

In another particular embodiment, the thermoplastic film is that where the polyamide is in an amount in the range of from 70-85% by weight. In another particular embodiment, the thermoplastic film is that where the polyamide is in an amount in the range of from 80 to 85% by weight.

The aliphatic homopolyamide can be selected from the group consisting of: polyamide 6 (PA6), polyamide 66 (PA66), polyamide 69, polyamide 610, polyamide 612 (PA612), polyamide 10 (PA10), polyamide 11 (PA11), and polyamide 12 (PA12), polyamide 46, polyamide 1010, and polyamide 1212.

The aliphatic Copolyamide can be selected from the group consisting of: Copolyamide 4/6 (PA4/6), Copolyamide 6/66 (PA6/66), Copolyamide 66/6 (PA66/6), Copolyamide 6/69 (PA6/69); Copolyamide 6/9 (PA6/9), Copolyamide 6/10 (PA6/10), copolyamide 4/10 (PA 4/10), Copolyamide 6/12 (PA6/12); a polyether-amides; a polyester amide; a polyether ester amide; a polyamide urethane; a poly(ether-block-amide).

The terpolyamide can be terpolyamide 6/66/12.

The partially aromatic polyamide can be selected from the group consisting of: polyamide 6-I where I is isophthalic acid, polyamide 6T where T is terephthalic acid, polyamide MXD6 which is a polycondensate of m-xylene diamine and adipic acid (nylon_MXD-6);

The partially aromatic copolyamide can be selected from the group consisting of: Copolyamide 6-1/6-T, Copolyamide 6/6-1, polyamide 4T/6T, polyamide 66/61 and mixtures thereof, wherein I is isophthalic acid and T is terephthalic acid.

In another particular embodiment, the thermoplastic film according to the invention is that where the polyamide is selected from the group consisting of aliphatic copolyamide selected from the group consisting of Copolyamide 4/6 (PA4/6), Copolyamide 6/66 (PA6/66), Copolyamide 66/6 (PA66/6), Copolyamide 6/69 (PA6/69); Copolyamide 6/9 (PA6/9), copolyamide 4/10 (PA 4/10), Copolyamide 6/10 (PA6/10); Copolyamide 6/12 (PA6/12), a polyether amide, a polyester amide, a polyether ester amide, a polyamide urethane, a poly(ether-block-amide), and terpolyamide 6/66/12 (e.g., Terpalex®). The nomenclature used to name homopolyamides and copolyamides is the one indicated in the norm ISO 1874-1.

In another particular embodiment, the thermoplastic film according to the invention is that where the polyamide is selected from the group consisting of: polyamide 6, polyamide 66, copolyamide 6/66, copolyamide 66, copolyamide 6/12, and tert-polyamide 6/66/12.

In another particular embodiment, the thermoplastic film according to the invention is that where the not cross-linked polyvinylpyrrolidone is in an amount in the range of from 13 to 25% by weight. In another particular embodiment, the thermoplastic film according to the invention is that where the not cross-linked polyvinylpyrrolidone is in an amount in the range of from 13 to 20% by weight. In another particular embodiment, the thermoplastic film according to the invention is that where the not cross-linked polyvinylpyrrolidone is in an amount in the range of from 13 to 16% by weight. These amounts provide low permeation and good transparency. In the case of bags, it contributes to the lack of water drops after sterilization of the bags.

In another particular embodiment, the thermoplastic film according to the invention is that where the plasticizer is selected from the group consisting of: xylitol, glycerin, sorbitol, 1,2-propyleneglycol, pentaerythritol, trimethyl propane, inositol, mannitol, triethanolamine, polyethylene glycol with a molecular weight of 200 to 10.000 amu (Dalton), mono and diglycol, trimethylolpropane, a mono-, di-, or triester of glycerol with carboxylic acids such as (C₃-C₁₂) alkanoic acids, formamide, acetamide, N,N-dimethylformamide, N,N-dimethylacetamide, and mixtures thereof. In another particular embodiment the plasticizer is selected from the group consisting of glycerin, xylitol, and sorbitol. In another particular embodiment, the thermoplastic film is that where the plasticizer is xylitol.

In another particular embodiment, the thermoplastic film is that where the plasticizer is in an amount in the range of from 1 to 3% by weight with respect to the total weight of the thermoplastic film. In another particular embodiment, the thermoplastic film is that where the plasticizer is in an amount of 2% by weight with respect to the total weight of the thermoplastic film.

In another particular embodiment, the thermoplastic film according to the invention is that which further comprises a further polymer or copolymer of polyolefin nature other than not cross-linked polyvinyl pyrrolidone and polyvinyl alcohol, with a melting point lower than 170° C.

In another particular embodiment, the thermoplastic film according to the invention is that wherein the further polymer or copolymer is selected from the group consisting of: polypropylene-grafted maleic anhydride PP-gMAH, polyethylene-grafted maleic anhydride (PEgMAH), ionomer of a copolymer of ethylene-acid (Surlyn), Methyl Methacrylate (MMA), Ethylene Methyl Acrylate EMA, and Ethylene Ethyl Acrylate EEA.

In another particular embodiment, the thermoplastic film according to the invention is that where the further polymer or copolymer is present in an amount in the range of from 5 to 15% by weight with respect to the weight of the thermoplastic film.

In a particular embodiment, the thermoplastic film according to the invention is that having a wall thickness in the range of from 20 to 400 μm. In another particular embodiment, the thermoplastic film according to the invention is that having a wall thickness in the range of from 20 to 80 μm. In another particular embodiment, the thermoplastic film has wall thickness in the range of from 40 to 70 μm. In another particular embodiment, the thermoplastic film has a wall thickness in the range of from 50 to 60 μm. These thermoplastic films are especially suitable for producing bags.

In another particular embodiment, the thermoplastic film has wall thickness in the range of from 200 to 300 μm. In another particular embodiment, the thermoplastic film has a wall thickness in the range of from 250 to 300 μm. These thermoplastic films are especially suitable for thermoformed packaging.

The thermoplastic film shows thermosealing resistance, good tear resistance, good puncture resistance, good tensile mechanical properties, and good water vapor permeability.

In a particular embodiment the thermoplastic film of the present invention has one layer. In another particular embodiment the thermoplastic film is a multilayered structure. In a particular embodiment, the thermoplastic film of the present invention is that having a multilayered structure which comprises a) at least one layer having a composition as defined above for the thermoplastic film of the present invention, this layer being a water vapor permeable layer P), and b) at least one heat sealable layer. Particularly, the layer having a composition as defined above for the thermoplastic film of the present invention comprises a) a polyamide in an amount in the range of from 60-85% by weight; b) not cross-linked polyvinyl pyrrolidone in an amount in the range of from 13 to 25% by weight; and c) a plasticizer in an amount in the range of from 1 to 4% by weight; wherein the amounts of each of the components are with respect to the total amount of components of the thermoplastic film; and one or more layers of another heat sealable layer.

In another particular embodiment, the thermoplastic films of the present invention have at least one water vapor permeable layer having a composition as defined above for the thermoplastic film of the present invention and at least one high oxygen barrier layer. The at least a high oxygen barrier layer comprises at least a high oxygen barrier polymer. For the purpose of the present invention high oxygen barrier polymer refers to a polymer showing oxygen transmission rate according <10 cc/m²·day·atm measured at 23° C. and 50% relative humidity according to DIN 53 380. Example of high oxygen barrier polymers are ethylene vinyl alcohol copolymers (EVOH), Polyvinylidene chloride or Polyxylylene adipamide (Nylon-MXD6).

In another particular embodiment, the thermoplastic films of the present invention have at least a water vapor permeable layer having a composition as defined above for the thermoplastic film of the present invention and at least one sealable layer and at least a high oxygen barrier layer.

The multilayer thermoplastic films according to the invention comprise at least two layers, produced by co-extrusion process providing mono or bi-oriented films or non-oriented films when extruded by blown or cast extrusion technology, exhibiting a water vapor transmission rate of at least 200 g/m²·day, preferably, a water vapor transmission rate (WVTR)≥250 g/m²·day, at 23° C. and 90/5, in accordance with DIN 53-122. In a particular embodiment, the water vapor transmission rate is in the range from 200 to 500 g/m²·day.

The multilayer thermoplastic films according to the invention may have from 2 to 13 layers, preferably from 2 to 11, more preferably from 2 to 9, and even more preferably from 2 to 7 layers. In a particular embodiment, the multilayer thermoplastic film has 2 layers. In another particular embodiment, the multilayer thermoplastic film has 3 layers.

In one embodiment thermoplastic film is that consisting of one heat sealable layer(S) and at least one permeable layer (P). In another embodiment multilayer thermoplastic films comprise two heat sealable layers, one heat sealable layer per each of the two external film side (S1 and S2) and at least one permeable layer (P) between the heat sealable layers.

In another embodiment multilayer thermoplastic films comprise one heat sealable layer(S) at least one permeable layer (P) and at least one high oxygen barrier layer (B) between the P and S layers.

In another embodiment multilayer film thermoplastic films comprise two heat sealable layers, one heat sealable layer per each of the two external film side (S1 and S2), at least one permeable layer (P) between the heat sealable layers and at least one high oxygen barrier layer (B)

The term heat sealable refers to a thermoplastic material that is able to be sealed with itself or another similar thermoplastic material using heat and pressure. The direct contact method of heat sealing utilizes a constantly heated die or sealing bar to apply heat to a specific contact area or path to seal or weld the thermoplastics together.

For the present purpose a heat sealable(S) layer is understood a layer comprising at least a polymer with a melting point lower than 170° C., preferably 150° C. It has good compatibility to the permeable layer. In a particular embodiment such good compatibility is with polyamide 6 (PA6). Preferably, this layer does not exhibit visual delamination between film S, P and B layers when film is submitted to a tensile test according to ISO 527/3, providing that multilayer thermoplastic film in which the water vapor permeation rate is at least 200 g/m²·day.

Examples of polymers of the S layer are Copolyamide 6/12 like for example Grilon CF 6S or Grilon CF 7 produced by EMS Grivory, thermoplastic elastomer ether-ester block copolymer like for example Arnitel series produced by DSM and thermoplastic elastomer polyether block amide PEBA like PEBAX series.

In another particular embodiment, the multilayer thermoplastic film consists of three or more layers where at least one of the inner layers comprises at least a pigment or at least one colorant and/or an UV block additive.

In another particular embodiment, the multilayer thermoplastic film consists of two or more layers where the outer film which will be the external side of the package comprise a water repellent additive like for example Ethylene Bis Stearamide (EBS).

Examples of film structures are as follows, but they are not limited to those of the following list: P/S; P1/P2/S where P1 and P2 comprise same polymer composition but different additive composition and/or additive content; S/P/S; S/B/P; S/P/B/P; and S/P/B/P/S.

In a particular embodiment, the thermoplastic film according to the present invention is, selected from the group consisting of: a thermoplastic film having a) at least one layer which has the composition as defined above b) at least one heat sealable layer; a thermoplastic film having a) at least one layer which has the composition as defined above, and b) at least one high oxygen barrier layer; and a thermoplastic film having a) at least one layer which has the composition as defined above, b) at least one high oxygen barrier layer; and c) at least one heat sealable layer.

In another particular embodiment, the thermoplastic film of the present invention is that which has a puncture force in the range of from 10 to 60 N measured by the method indicated in the examples section. In another particular embodiment, the packaging is that which has a puncture force in the range of from 15-40 N. In another particular embodiment, the packaging is that which has a puncture force in the range of from 40-60 N. In a particular embodiment, the thermoplastic film is a non-oriented film which has a puncture force in the range of from 15N at 30 μm film thickness to 52 N at 100 μm. In another particular embodiment, the thermoplastic film is a bi-oriented film with a puncture force in the range of 29 N at 28-30 μm.

In another particular embodiment, the thermoplastic film is that which has a sealing strength in the range of from 5-30 N measured by the method indicated in the examples section. In another particular embodiment, the packaging is that which has a sealing strength in the range of from 20 to 30N. In another particular embodiment, the packaging is that which has a sealing strength in the range of from 14 to 35 N.

In another particular embodiment, the thermoplastic film is that which has a machine direction tensile strength in the range of from 50 to 180 MPa measured by the method indicated in the examples section. In another particular embodiment, the packaging is that which has a machine direction tensile strength in the range of from 60 to 160 MPa.

In another particular embodiment, the thermoplastic film is that which has a transversal direction tensile strength in the range of from 40 to 180 measured by the method indicated in the examples section. In another particular embodiment, the packaging is that which has a transversal direction tensile strength in the range of from 50 to 160 MPa.

In another particular embodiment, the thermoplastic film is that which has a machine direction tensile elongation in the range of from 100 to 450% measured by the method indicated in the examples section. In another particular embodiment, the packaging is that which has a machine direction tensile elongation in the range of from 130 to 500%.

In another particular embodiment, the thermoplastic film is that which has a transversal direction tensile elongation in the range of from 60 to 500% measured by the method indicated in the examples section. In another particular embodiment, the packaging is that which has a transversal direction tensile elongation in the range of from 80 to 475%.

In another particular embodiment, the thermoplastic film is that which has a machine direction tearing maximum force in the range of from 0.1-3 N measured by the method indicated in the examples section. In another particular embodiment, the packaging is that which has a machine direction tearing force in the range of from 0.2 to 2 N. In another particular embodiment, the packaging is that which has a machine direction tearing force in the range of from 0.2 to 0.9 N.

In another embodiment, the thermoplastic film is that which has a transversal direction tearing maximum force in the range of from 0.1-4.0 N measured by the method indicated in the examples section. In another particular embodiment, the packaging is that which has a transversal direction tearing force in the range of from 0.2 to 2.5 N.

In a particular embodiment, the thermoplastic film is that which has a water vapor transmission rate of at least 250 g/m² per day measured for the film before sterilization by the method detailed in the examples section (WVTR, at 23° C. and 90/5 HR % according to a DIN 53-122 using a Permatran equipment from Mocon and measured before sterilization). In another particular embodiment, the packaging is that which has a water vapor transmission rate of at least 400 g/m² per day. In another particular embodiment, the packaging is that which has a water vapor permeability of at least 200-500 g/m² per day.

In another particular embodiment, the thermoplastic film is that which has an oxygen transmission rate lower than 50 cc/m²·day.

All the particular or preferred features of the different embodiments disclosed above for the films are disclosed either alone or in any in combination thereof.

The films may be prepared by a process comprising the following steps: a) melt blending the individual components that will form the film which are at least a polyamide in an amount in the range of from 60 to 85% by weight, not cross-linked PVP in an amount in the range of from 13 to 25% by weight, and a plasticizer in an amount in the range of from 1 to 4% by weight, wherein the amounts of each of the components are with respect to the total amount of components of the film; b) extruding the mixture by a process selected from a mono-orientation process, a bi-orientation process, a blow film not oriented extrusion process, and a cast film not oriented extrusion process; and c) in case of oriented film process, thermally anneal the film.

The films of the present invention may be prepared by a process comprising the following steps: a) melt blending the individual components that will form the film which are at least a polyamide in an amount in the range of from 60 to 85% by weight, not cross-linked PVP in an amount in the range of from 13 to 25% by weight, and a plasticizer in an amount in the range of from 1 to 4% by weight, and an additive selected from the group consisting of a water repellent, a slipping agent, an additive for providing color, anti-blocking agent, a mineral filer, a nucleating agent, a UV block additive, a surfactant, and mixtures thereof; wherein the amounts of each of the components are with respect to the total amount of components of the film; b) extruding the mixture by a process selected from a mono-orientation process, a bi-orientation process, a blow film not oriented extrusion process, and a cast film not oriented extrusion process; and c) in case of oriented film process, thermally anneal the film.

In a particular embodiment the components are melt blended by means of a twin-screw extruder, at maximum setting extruder temperatures from 210 to 270° C. preferably 220-240° C. Molten blend mass flowing through out the die extruder holes as filaments are cooled and cut into pellet shape.

In another particular embodiment, the film is produced by the process known as double-bubble process.

The preparation process helps to provide the observed dimensional stability at 30-150° C. (heat shrink≤2%, ≤1%, or even≤0.5%). Thermo shrinkage is determined by soaking a sample of known and measured length (longitudinal or extrusion direction) and width (transversal to extrusion direction) into water vessel at 80° C. for 1 minute. After one minute sample is removed from water and its length and width is measured again.

Thermo shrinkage is defined as (Xₒ−X₁)/Xₒ*100 where Xₒ and X₁ are the dimension measured before and after soaking in hot water respectively. Thermo shrinkage is determined in both directions, longitudinal and transversal direction.

The film of the present invention may also be defined by its preparation process. Thus, a film obtainable by a process comprising a) melt blending the individual components that will form the film which are at least a polyamide in an amount in the range of from 60-85% by weight, not cross-linked PVP in an amount in the range of from 13 to 25% by weight, and a plasticizer in an amount in the range of from 1 to 4% by weight, and an additive selected from the group consisting of a water repellent, a slipping agent, an additive for providing color, anti-blocking agent, a mineral filer, a nucleating agent, a UV block additive, a surfactant, and mixtures thereof; wherein the amounts of each of the components are with respect to the total amount of components of the film; b) extruding the mixture by a process selected from a mono-orientation process, a bi-orientation process, a blow film not oriented extrusion process, and a cast film not oriented extrusion process; and c) in case of oriented film process, thermally anneal the film, is also considered part of the invention. The film may be mono or bi-oriented or a blow or cast extrusion film which are not oriented.

It is also part of the invention, a packaging comprising one or more single or multilayer thermoplastic films as defined above, joined by at least one seal seam, or adhesive bonded. The packaging may be a bag or pouch. The packaging is suitable for the sterile packaging of objects for medical, pharmaceutical or cosmetical applications. In a preferred embodiment, the packaging is made of only one material having all sides transparent or translucid. They have the advantage of easiness to recycling versus medical materials such as dissimilar medical bags made of paper/plastic or Tyvek/plastic.

The packaging of the present invention is suitable for sterilizing objects inside by means of water vapor or ethylene oxide. The packaging is also microbiologically impermeable.

In a particular embodiment, the packaging is that in which the film is adhesive bonded.

The adhesive may be applied on the surfaces of the film using known processes such as spraying or roll coating. The packaging is assembled by uniting two films seams.

In another particular embodiment, the packaging is that in which the joint or joints are made by heat sealing.

The thermoplastic films and the packaging of the present invention are dimensionally stable at a temperature of in the range of from 30 to 150° C. In a particular embodiment, the packaging of the present invention is that in which the thermoplastic film(s) has a heat shrinkage of less than 2% in transverse and longitudinal direction. In another particular embodiment, the packaging is that in which the thermoplastic film(s) has a heat shrinkage of less than 1% in transverse and longitudinal direction. In another particular embodiment, the packaging of the present invention is that in which the thermoplastic film(s) has a heat shrinkage of less than 0.5% in transverse and longitudinal direction.

In a particular embodiment, the packaging is that which has a vapor permeation to oxygen <100 cc/m²·day. In another particular embodiment, the packaging is that which has a vapor permeation to oxygen <50 cc/m²·day. This property is especially suitable for packaging products sensible to oxidation such as sterilized collagen.

All the particular features of the different embodiments disclosed above for the packaging are disclosed either alone or in any combination thereof.

In a particular embodiment, the packaging as defined above, further comprises one or more objects or materials for medical, pharmaceutical or cosmetic applications.

It is also part of the invention, a process for preparing the packaging as defined above. The packaging may be prepared as follows.

In a particular embodiment, the packaging is assembled by uniting two flat films according to the present invention and may be joined, for instance, by heat sealing three seams by means of sealant heat bands at temperature between 140° C. and 220° C. during 0.1 and 5 s and a pressure at least 0.1 N/mm².

In another embodiment, the films according to the present invention are tubular films, i.e., seamless, and one end of the tube is heat sealed.

In another particular embodiment, a flat film by a commonly known “flow pack” process” is continually conformed to a tubular shape, item to be packed is placed in it, and film is heat sealed longitudinally, by overlapping inner side to outer side of film or the inner to inner side of the film, heat transversally to close hermetically the package and cut to desired package length.

In another particular embodiment, a film is depth thermoformed, e.g., forming a tray, and another flat film is placed on the thermoformed body and heat sealed to the edges of the thermoformed body.

The film according to the invention can also be conformed and heat sealed in a package by dosing on the surface a heat seal coating adhesive.

Examples of those adhesive are heat sealable coatings of acrylic and co-polymers of acrylate solvent or ethylene vinyl acetate (EVA) solvent or water-based adhesives. Dosing of hotmelt adhesives is also possible. Examples are EVA and Polyurethane based adhesive hotmelts. Dosing adhesives or glues enables a package defined by a film according to the invention joined to a non-thermoplastic film material (like paper, aluminium, inorganic coatings, etc.) or no polyamide compatible polymers (like polyolefins etc.). Examples of those adhesive are heat sealable coatings of acrylic and co-polymers of acrylate solvent based or ethylene vinyl acetate (EVA) solvent or water-based adhesives. Dosing of hotmelt adhesives is also possible. Examples are copolymers of ethylene vinyl (EVA), acrylate-polyamide copolymers or Polyurethane based adhesive hotmelts. Dosing or coating of adhesives or glues enables a package defined by a film according to the invention joined to a non-thermoplastic film materials (like paper, aluminium, inorganic coatings, etc.) or non-polyamide compatible polymers (like polyolefins etc.).

The packaging as defined above can be used for several purposes.

One of the possible uses of the packaging is to sterilize objects or materials by means of water vapor or ethylene oxide. It may be useful for medical, pharmaceutical, or cosmetic applications material such as sealable bags or thermoformed packaging where films of the present invention can be the thermoformed bottom and/or the lip closing the packaging.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim and shall not be construed as limiting the scope of the claim. Furthermore, the present invention covers all possible combinations of particular and preferred embodiments described herein.

### EXAMPLES

Measuring methods used for determining the following parameters of the thermoplastic films and bags of the present invention.

Water vapor transmission rate was determined in accordance with DIN 53-122 at 23° C. 90/5% relative humidity using a Permatran-WR permeation analyzer by Mocon. 90/5% relative humidity is the % humidity in the nitrogen stream flowing continuously above/below respectively the sample film to be evaluated in the water vapor permeation equipment which for the present invention is Permatran from Mocon. It can also be expressed as 85% relative humidity which is the difference or gradient between the two streams.

Oxygen transmission rate was analyzed in accordance with DIN 53 380 at 23° C. and 50% relative humidity using an Ox-Tran® permeation analyzer by Mocon.

MD and TD tensile strength and MD and TD tensile elongation were measured in accordance with ISO 527-3 using a universal testing equipment by Instron 3365.

Maximum puncture force was assessed in accordance with internal test method using a universal testing equipment Instron 3365 mn.
Conditioning of sample at 23° C.@50% RH for 8 hours (at least).
Size of sample: diameter 80 mm
Cell load: 1000N
Speed: 500 mm/min
Blunt tip. flat tip with diameter of 2.5 mm.

Sample with diameter of 80 mm is mounted in sample holder. Tip is moved down in order to gently touch the surface of measured film with initial force≤0.5N. Measurement is started under conditions described above.

Max force (N) for puncture is recorded. At least 6 measurements per sample are carried out.

MD and TD tearing force was assessed by internal method using a universal testing equipment Instron 3365
Size of sample: length 80 mm
Cell load: 100N
Speed: 100 mm/min
Grip distance: 40 mm

Sample with length of 80 mm and width of 30 mm is partially cut on half (length of partial cut=40 mm, width of cut=15 mm-see attached picture) and it is mounted between clamps (initial cut is parallel with direction of elongation) where grip distance is 40 mm. Initial force (elongation=0%) should be between 0.1N and 0.5N.

Conditioning of sample at 23° C.@50% RH for 8 hours (at least).

Measurement is started under conditions described above.

Maximal force (N) for tearing is recorded. At least 6 measurements per sample are carried out. Measurements are done at both direction (MD and TD).

Heat seal strength was determined in accordance with internal method using a universal testing equipment Instron 3365
Size of sample: length 120 mm, width of 25 mm
Cell load: 1000N
Speed: 500 mm/min
Grip distance: 40 mm

Initially, samples are sealed by using our sealing machine (KOPP model SGPE 20). Sealing machine is equipped with two sealing heating bars (upper and lower). Both heating bars were used for our sealing experiment under following conditions:
sealing force=450N
heating/sealing time=1 s
cooling time=5 s

Sealed samples were cut to prepare stripes with length of 120 mm and width of 25 mm. Samples are mounted between clamps with grip distance of 40 mm (seal is located in the middle distance between of clamps and perpendicular to the direction of elongation). Initial force (elongation=0%) is set between 0.5N and 1.0N.

Measurement is started under conditions described above.

Max force (N) of seal/seal strength (N) is recorded. At least 6 measurements per sample are carried out.

Wall thickness of the thermoplastic film is measured with a micrometer.

Water vapor uptake is measured by internal gravimetric test method: bags consisting of (tubular) film according to the invention and films according to comparative example are conformed by heat sealing all bag edges except one through which Silica gel is introduced. After that open edge bags are heat sealed and bags re weighted (w0). Sample bags with the silica are soaked into an autoclave at 120° C. for 1 h after which bags are removed from the autoclave, water on surface is removed with a paper and finally bags are weighted again (w1).

Water uptake is calculated as (w₁−W₀)/bag surface area/day and it is expressed as g/m²·day

### Example 1: Production of Copolyamide 6/66—PVP Compound

Plasticizer (Xylitol E967, food grade, crystals 10-30 mesh, Brenntag) and polyvinyl pyrrolidone (Kollidon K30 by BASF) were fed in the main feeder of a Co-rotating Twin-Screw-Extruder, Leistritz GL27: Ø=27 mm, L/D=36. Co-Polyamide 6/66 UBE NYLON 5024 were dosed by a gravimetric side feeder to the extruder. Total feeding rate was 15 kg/h and screws rpm 300. All ingredients were thermally blended and plasticized at melt temperature (actual mass temperature measured with a thermocouple probe in the extruder) of 230-235° C. to obtain through the die filaments which were cooled down previously to be pelletized. Dosing ratio of each ingredient provides 82% Copolyamide 6/66, 16% polyvinyl pyrrolidone and 2% xylitol compound composition.

### Example 2: Production of a Bioriented Mono Layer Film-Formulation 82% CoPA 6/66 UBE 5024B 16% PVP Kollidon K30; 2% Xylitol

Co-polyamide 6/66—PVP obtained in Example 1 were fed in a single screw 30 mm extruder plasticizing and homogenizing the individual recipe components and forming the melt in an extrusion head into a so-called primary tube. The primary tube was rapidly quenched by means of cold water and pre-heated by hot water, bi-axially oriented, and thermally annealed before winding. This process is well-known as the double-bubble-process with an annealing step or also as triple bubble process. The primary tube was stretched by a factor of 1.9 in machine direction (MD) and by a factor of 2.9 in transversal direction (TD). After being stretched film was heat annealed at 200° C. Final wound tubular film had a wall thickness of 25 μm and 1-4% thermal shrinkage in its longitudinal and transversal direction. Tubular film was opened to get two flat films by cutting with two knifes both flat tube edges. Film obtained exhibits acceptable transparency and no odour.

### Example 3: Production of a Monolayer Blown Film—Formulation 82% CoPA 6/66 UBE 5024B 16% PVP Kollidon K30; 2% Xylitol

Same compound obtained in Example 1 was fed together with 1000 ppm of EBS in a 20 mm single screw extruder where material was thermally plasticized at a melt temperature of 220-225° C. The extrudate melt was fed through a tubular die head of 80 mm diameter and 1 mm die gap and immediately inflated up using air inside of formed bubble. Film bubbles were cooled down by chill air and collected and pulled by nip rolls located up and vertically from the die head, cut its lateral edges into two flat films using knifes and finally winding up the film.

30, 50 and 100 μm wall thickness film was obtained with 0-0.5% and 1-2% thermal shrinkage in its longitudinal and transversal direction with good optical properties and no major issues. Film obtained exhibits acceptable transparency and no odour.

### Example 4: Comparison of the Properties of the Permeable and Monolithic Plastic Films of the Present Invention with Respect to Paper Sealed to Plastic PET/PP No Permeable Film Bag and with Film Formulations of Polyamides with Polyvinyl Alcohol

### Samples:

Example 2: Bioriented monolayer film—formulation 82% Copolyamide 6/66 UBE 5024B 16% PVP kollidon K30; 2% Xylitol
Wall film thickness 25 μm

Heat sealing strength of film according to Example 2 was performed by heat sealing film to itself and evaluate it in accordance with internal method using a universal testing equipment Instron 3365

Bag water uptake test: bags consisting of (tubular) film in accordance with example 2 are conformed by heat sealing all bag edges except one through which Silica gel is introduced.
Example 3: Monolayer blown film (not oriented)—formulation 82% Copolyamide 6/66 UBE 5024B 16% PVP kollidon K30; 2% Xylitol and 1000 ppm EBS.

Heat sealing strength of film according to example 3 was performed by heat sealing film to itself and evaluate it in accordance with internal method using a universal testing equipment Instron 3365.

Bag water uptake test: bags consisting of (tubular) film in accordance with Example 3 are conformed by heat sealing all bag edges except one through which Silica gel is introduced.
Comparative Example 1A: commercial reference medical grade bag made of paper sealed to no permeable Polyethylene Terephthalate/bi-oriented polypropylene (PET/BoPP) laminated 45 μm thickness plastic film.
Comparative Example 1B: Paper film from the commercial retorting bag.
Comparative Example 1C: PET/PP film from the commercial retorting bag.
Comparative Example 1D: Bag produced by heat-sealing medical paper of the commercial medical bag to the film of example 2.
Comparative Example 2: Bioriented mono layer film-formulation containing a blend of 71% Copolyamide 6/66 UBE 5033B+20% thermoplastic polyvinyl alcohol+4% Try-Ethylene Glycol. This polyvinyl alcohol polymeric resin product is known and commercially available as Mowiflex® TC 232 produced by Kuraray Specialties Europe GmbH and is described and claimed in the International Patent Application No. WO2003020823.

Heat sealing strength of film according to Example 3 was performed by heat sealing film to itself and evaluate it in accordance with internal method using a universal testing equipment Instron 3365.

Bag water uptake test: bags consisting of (tubular) film in accordance with comparative Example 2 are conformed by heat sealing all bag edges except one through which Silica gel is introduced.

The properties of the different sample films were measured before sterilization. The results are shown below.

TABLE 1
Results:

| | Film Wall | | | |
|---|---|---|---|---|
| | thickness | Film WVTR | Film Puncture | Sealing strength |
| | (μm) | (g/m² · day) | Force (N) | (N) |
| Sample Description | Micrometer | DIN 53 122 | blunt tip, | Viscofan |
| | | 23° C., | 500 mm/min, | method as |
| | | 90/5% RH | conditioned | described herein |
| Example 2 | 25 | 356 | 29 | 26 |
| Example 3 | 31 | 300 | 15 | 25 |
| Comparative | | | | 7 |
| Example 1A | | | | |
| Comparative | 76 | >2000 | 14 | |
| Example 1B | | | | |
| Comparative | 43 | 2.6 | 23 | |
| Example 1C | | | | |
| Comparative | | | | 8 |
| Example 1D | | | | |
| Comparative | 17 | 220 | 29 | 24 |
| Example 2 | | | | |

| | Film | Film | | | |
|---|---|---|---|---|---|
| | tensile | tensile | Film | | Bag water |
| | strength | elongation | Tearing | Film OTR | uptake |
| | (Mpa) | (%) | Force (N) | (cc/m² · day) | (g/m² · day) |
| Sample | ISO | ISO | 100 mm/min, | DIN 53 | Internal |
| Description | 527-3 | 527-3 | conditioned | 380 23° C., | gravimetric |
| | MD/TD | MD/TD | MD/TD | 50% RH | method as |
| | | | | | described herein |
| Example 2 | 152/159 | 137/82 | 0.2/0.1 | 29 | 7548 |
| Example 3 | 71/63 | 421/470 | 0.8/1.3 | 31 | 7640 |
| Comparative | | | | | 4844 |
| Example 1A | | | | | |
| Comparative | 99/46 | ′5/9 | 1.1/1.2 | 1000 | |
| Example 1B | | | | | |
| Comparative | 80/78 | 97/89 | 0.7/3.9 | 85 | |
| Example 1C | | | | | |
| Comparative | | | | | 9453 |
| Example 1D | | | | | |
| Comparative | 181/300 | 106/112 | 0.1/0.1 | 30 | 7640 |
| Example 2 | | | | | |

Comparative Example 2 shows strong acetic odor. In addition, it exhibits opalescence effect after soaking film in wet heat. Conversely, the films of Examples 2 and 3 do not present odor, neither opalescence.

The results show that the films of the present invention are permeable plastic with outstanding mechanical properties, in particular, in the case of biorientation. The films show good sealing strength, good puncture resistance, good permeability, in all cases better than the comparative examples. Down gauge film leads to potential cost saving and more ecological friendly material.

A blown no oriented film also shows good physical properties and permits higher efficiency and production cost savings when comparing to bi-orientation extrusion process.

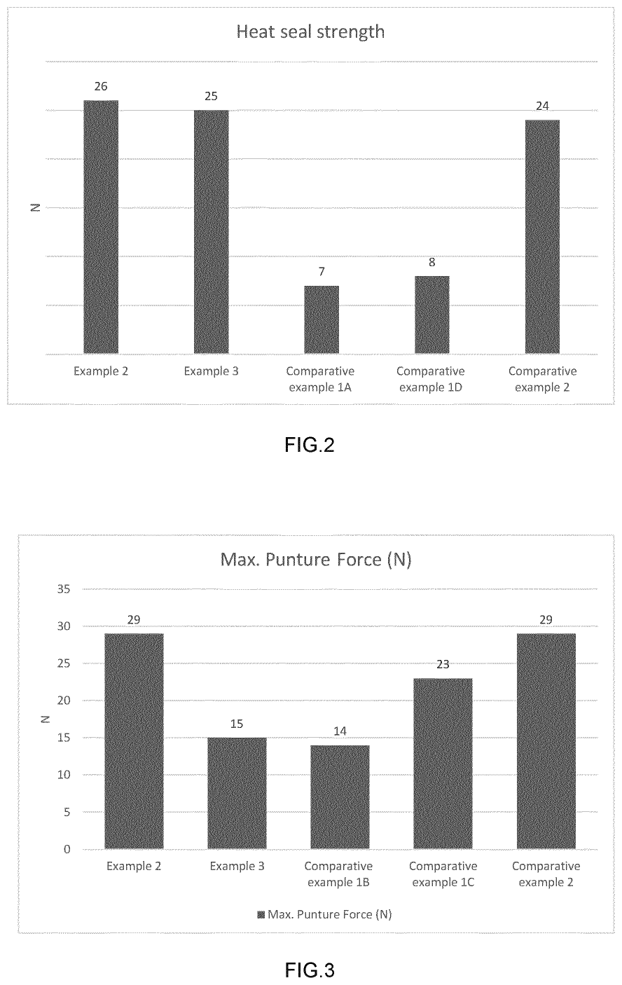

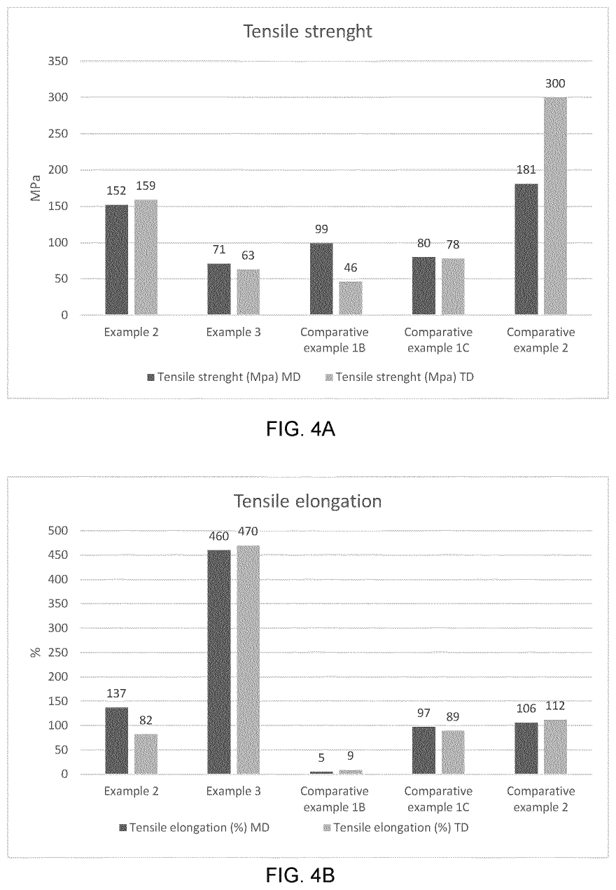

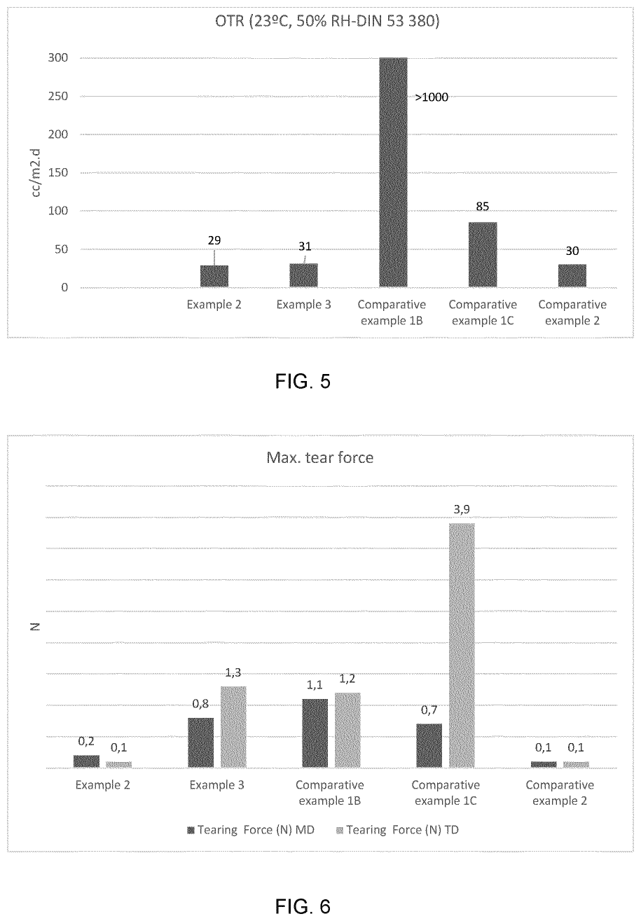

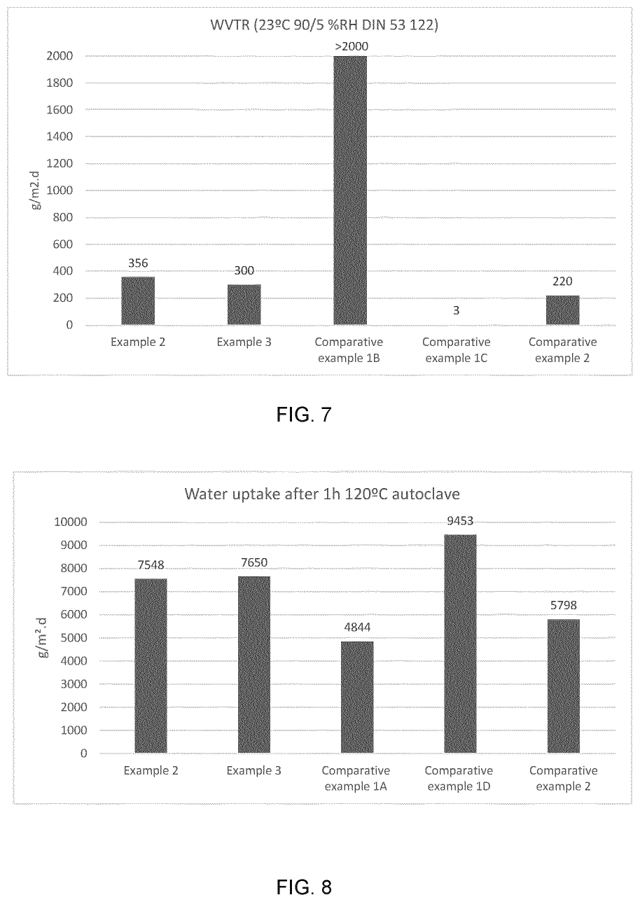

The films of the invention show high sealing strength (see FIG. 2), high puncture resistance (see FIG. 3), good tensile properties (FIGS. 4A and 4B), low Oxygen permeability (FIG. 5), tear strength (see FIG. 6), high water vapor transmission rate of films (see FIG. 7) and bags water uptake after autoclave treatment (see FIG. 8). Films and bags in accordance with the invention show higher water vapor permeation and higher water uptake after being soaked into an autoclave which reveal films and bags of the present invention are at least as effective as comparative example 1 bags used commercially nowadays. In addition, bags according to the invention shows acceptable optical properties after autoclaving.

WVTR of paper, Comparative example 1B is >2000 g/m² per day which is a value that fall outside the detection limit of the equipment.

### Example 5: Vapor Sterilization Assay of the Packaging

Water vapor uptake is measured by internal gravimetric test method: bags consisting of (tubular) film according to the invention and films according to comparative example are conformed by heat sealing all bag edges except one through which, Silica gel with orange moisture indicator-reference 1.01969.5000 Sigma-millipore-dried in a vacuum oven 4 h at 130° C. is introduced. After that, open edge bags are heat sealed and bags re weighted (w0). Sample bags with the silica are soaked into an autoclave at 120° C. for 1 h after which bags are removed from the autoclave, water on surface is removed with a paper and finally bags are weighted again (w1).

Water uptake is calculated as (w1-w0)/bag surface area/days and it is expressed as g/m²·day.

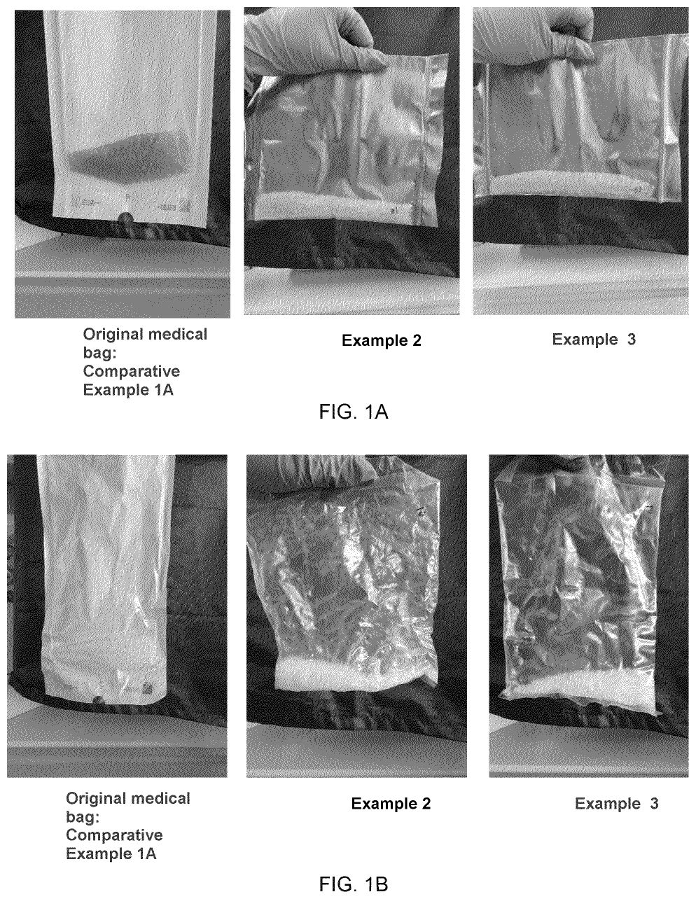

A comparison between packaging according to the invention (Examples 2 and 3) and bags according to the prior art can be seen in the figures. FIG. 1A and FIG. 1B show respectively bags before and after sterilization.

FIG. 8 shows test results of water uptake. Films and containers of the invention are transparent and are at least as effective as the comparative examples that are used commercially nowadays.

### Example 6 Packaging Sterilization Assay by Ethylene Oxide (EO) of the Packaging

Collagen film (control) was analyzed determining number of colonies of microorganisms per gram of sample (collagen film produced by Viscofan).

### Microorganisms Analyzed:

Mesophilic aerobic microorganisms: Bacteria, yeasts and molds that grow aerobically.
Fungi.—aerobic mesophilic filamentous microorganism that grows diffusely on Petrifilm Rapid Yeast and Mold, usually blue in color, but can also have its natural coloration (black, green).
Yeast—mesophilic aerobic microorganism that produces small, well-defined, blue-green colonies on Petrifilm Rapid Yeast and Mold.

Collagen films were packed into a packaging consisting of heat sealed films according to example 3. The two opened seams of tubular film according to example 3 were heat sealed. As Comparative example was used a bag according to comparative example 1A.

Collagen films into sealed packaging were sterilized with ethylene oxide and after the process the number of colonies were counted.

Results (see table 2) show ethylene oxide effectively sterilize objects into a packaging according to the invention. Collagen film packed into the packaging according to the invention after being treated with ethylene oxide shows a significant CFU/g decrease (Colony-forming Unit per gram) from values >10\hat{ }2 to values <10 CFU/g. In addition, since it is monolithic and exhibits low permeability to oxygen, it provides an additional contamination security to sterilized and packed objects during its post sterilization storage compared to objects sterilized into packaging having porosity or high permeation to oxygen.

TABLE 2

| | Total mesophile | Fungi | Yeast |
|---|---|---|---|
| Sample | aerobes (cfu/g) | (cfu/g) | (cfu/g) |
| Collagen Film -Control | 1.60E+02 | 3.20E+02 | 1.10E+02 |
| Collagen Film Packed | <10 | <10 | <10 |
| into bag comprising | | | |
| film example 3 | | | |
| Collagen Film packed | <10 | <10 | <10 |
| into bag comparative | | | |
| example 1A | | | |

### Ethylene Oxide Process Procedure:

a) Preheating: chamber is heated up to 55° C. for 60 min.
b) Conditioning: This phase includes vacuum, tightness test, vacuum, steam injections and absorption until reaching 55° C. and 98% relative humidity and subsequent vacuum, before drilling the EO cartridge. Total conditioning time was between 47±5 min.
c) Exposure to EO gas: perforation of the EO cartridge and maintenance of the temperature, 55° C., Relative humidity (98%) and pressure conditions for 45 minutes.
d) EO extraction and aeration:

At the end of the exposure phase, a vacuum is created up to a pressure of ≤250 mbar abs for the extraction of the EO gas with subsequent entry of filtered air into the chamber up to a pressure not exceeding 950 mbar abs.

Aeration: Air extraction with simultaneous air intake, maintaining the chamber pressure at a pressure not higher than 950 mbar abs. and a duration of 48 hours.

### Clause

1. A thermoplastic film comprising:
a) a polyamide in an amount in the range of from 60-85% by weight;
b) not cross-linked polyvinyl pyrrolidone in an amount in the range of from 13 to 25% by weight; and
c) a plasticizer in an amount in the range of from 1 to 4% by weight;
wherein the amounts of each of the components are with respect to the total amount of components of the thermoplastic film.

2. The thermoplastic film according to clause 1, further comprising an additive selected from the group consisting of a water repellent, a slipping agent, an additive for providing color, anti-blocking agent, a mineral filer, a nucleating agent, a UV block additive, a surfactant, and mixtures thereof.

3. The thermoplastic film according to any of the clauses 1-2, comprising at least an additive, wherein the additive is a waster repellent selected from the group consisting of a N,N′—(C₁₆-C₂₂)-alkylenebis amide and a triglyceride ester of a fatty acid ranging from C₈-C₃₀.

4. The thermoplastic film according to any of the clauses 1-3, wherein the polyamide is selected from the group consisting of: aliphatic homopolyamide, aliphatic copolyamide, partially aromatic homopolyamide, partially aromatic copolyamide, ter-polyamide, and mixtures thereof.

5. The thermoplastic film according to any of the clauses 1-4, wherein the polyamide is selected from the group consisting of: polyamide 6, polyamide 66, copolyamide 6/66, copolyamide 66/6, copolyamide 6/12, and terpolyamide 6/66/12.

6. The thermoplastic film according to any of the clauses 1-5, wherein the polyamide is in an amount in the range of from 70-85% by weight.

7. The thermoplastic film according to any of the clauses 1-6, wherein the not cross-linked polyvinylpyrrolidone is in an amount in the range of from 13 to 20% by weight.

8. The thermoplastic film according to any of the clauses 1-7, wherein the plasticizer is selected from the group consisting of glycerin, xylitol, and sorbitol.

9. The thermoplastic film according to any of the clauses 1-8, selected from the group consisting of:
A thermoplastic film having a) at least one layer which has the composition as defined in any of the claims 1-8, and b) at least one heat sealable layer;
A thermoplastic film having a) at least one layer which has the composition as defined in any of the claims 1-8, and b) at least one high oxygen barrier layer; and
A thermoplastic film having a) at least one layer which has the composition as defined in any of the claims 1-8, b) at least one high oxygen barrier layer; and c) at least one heat sealable layer.

10. The thermoplastic film according to any of the clauses 1-9, which has a water vapor transmission rate WVTR≥250 g/m²·day, at 23° C. and 90/5.

11. The film according to any of the clauses 1-10, which has an oxygen transmission rate lower than 50 cc/m²·day.

12. A packaging comprising a single or multilayer thermoplastic film as defined in any of the clauses 1-11, joined by at least one seal seam, or adhesive bonded.

13. The packaging according to clause 12, further comprising one or more objects or materials inside the packaging for medical, pharmaceutical or cosmetic applications.

14. A process for preparing the thermoplastic film as defined in any of the clauses 1-11, which comprises:
a) melt blending the individual components that will form the thermoplastic film which are at least a polyamide in an amount in the range of from 60-85% by weight, not cross-linked PVP in an amount in the range of from 13 to 25% by weight, and a plasticizer in an amount in the range of from 1 to 4% by weight, wherein the amounts of each of the components are with respect to the total amount of components of the thermoplastic film;
b) extruding the mixture by a process selected from a mono-orientation process, a bi-orientation process, a blow film not oriented extrusion process, and a cast film not oriented extrusion process; and
c) in case of oriented film process, thermally anneal the film.

15. Use of a packaging as defined in clause 12, to sterilize objects or materials by means of water vapor or ethylene oxide.

### CITATION LIST

### Patent Literature

CA1314265C
US20200407142A1
WO2006/012482
EP1380212B1
WO02078455A1

What is claimed is:

1. A thermoplastic film comprising:
a) a polyamide in an amount in the range of from 60-85% by weight;
b) not cross-linked polyvinyl pyrrolidone in an amount in the range of from 13 to 25% by weight; and
c) a plasticizer in an amount in the range of from 1 to 4% by weight;
wherein the amounts of each of the components are with respect to the total amount of components of the thermoplastic film further comprising an additive selected from the group consisting of a water repellent, a slipping agent, an additive for providing color, anti-blocking agent, a mineral filer, a nucleating agent, a UV block additive, a surfactant, and mixtures thereof.

2. The thermoplastic film according to claim 1, comprising at least an additive, wherein the additive is a water repellent selected from the group consisting of a N,N′—(C₁₆-C₂₂)-alkylenebis amide and a triglyceride ester of a fatty acid ranging from C₈-C₃₀.

3. The thermoplastic film according to claim 1, wherein the polyamide is selected from the group consisting of: aliphatic homopolyamide, aliphatic copolyamide, partially aromatic homopolyamide, partially aromatic copolyamide, ter-polyamide, and mixtures thereof.

4. The thermoplastic film according to claim 1, wherein the polyamide is selected from the group consisting of: polyamide 6, polyamide 66, copolyamide 6/66, copolyamide 66/6, copolyamide 6/12, and terpolyamide 6/66/12.

5. The thermoplastic film according to claim 1, wherein the polyamide is in an amount in the range of from 70-85% by weight.

6. The thermoplastic film according to claim 1, wherein the not cross-linked polyvinylpyrrolidone is in an amount in the range of from 13 to 20% by weight.

7. The thermoplastic film according to claim 1, wherein the plasticizer is selected from the group consisting of glycerin, xylitol, and sorbitol.

8. The thermoplastic film according to claim 1, selected from the group consisting of:
a thermoplastic film having a) at least one layer which has the composition as defined in claim 1, and b) at least one heat sealable layer which is understood a layer comprising at least a polymer with a melting point lower than 170° C.;
a thermoplastic film having a) at least one layer which has the composition as defined in claim 1, and b) at least one high oxygen barrier layer showing oxygen transmission rate according <10 cc/m²·day·atm measured at 23° C. and 50% relative humidity according to DIN 53 380; and
a thermoplastic film having a) at least one layer which has the composition as defined in claim 1, b) at least one high oxygen barrier layer showing oxygen transmission rate according <10 cc/m²·day·atm measured at 23° C. and 50% relative humidity according to DIN 53 380; and c) at least one heat sealable layer.

9. The thermoplastic film according to claim 1, which has a water vapor transmission rate WVTR≥250 g/m²·day, at 23° C. and 90/5.

10. The film according to claim 1, which has an oxygen transmission rate analyzed in accordance with DIN 53 380 at 23° C. and 50% relative humidity using an Ox-Tran® permeation analyzer lower than 50 cc/m²·day.

11. A packaging comprising a single or multilayer thermoplastic film comprising:
a) a polyamide in an amount in the range of from 60-85% by weight;
b) not cross-linked polyvinyl pyrrolidone in an amount in the range of from 13 to 25% by weight; and
c) a plasticizer in an amount in the range of from 1 to 4% by weight;
wherein the amounts of each of the components are with respect to the total amount of components of the thermoplastic film,
joined by at least one seal seam, or adhesive bonded.

12. The packaging according to claim 11, further comprising an additive selected from the group consisting of a water repellent, a slipping agent, an additive for providing color, anti-blocking agent, a mineral filer, a nucleating agent, a UV block additive, a surfactant, and mixtures thereof.

13. The packaging according to claim 11, further comprising one or more objects or materials inside the packaging for medical, pharmaceutical or cosmetic applications.

14. A process for preparing the thermoplastic film as defined in claim 1, which comprises:
a) melt blending the individual components that will form the thermoplastic film which are at least a polyamide in an amount in the range of from 60-85% by weight, not cross-linked PVP in an amount in the range of from 13 to 25% by weight, and a plasticizer in an amount in the range of from 1 to 4% by weight, and an additive selected from the group consisting of a water repellent, a slipping agent, an additive for providing color, anti-blocking agent, a mineral filer, a nucleating agent, a UV block additive, a surfactant, and mixtures thereof; wherein the amounts of each of the components are with respect to the total amount of components of the thermoplastic film;
b) extruding the mixture by a process selected from a mono-orientation process, a bi-orientation process, a blow film not oriented extrusion process, and a cast film not oriented extrusion process; and
c) in case of oriented film process, thermally anneal the film.

15. Use of a packaging as defined in claim 12, to sterilize objects or materials by means of water vapor or ethylene oxide.

16. The thermoplastic film according to claim 2, wherein the polyamide is selected from the group consisting of: aliphatic homopolyamide, aliphatic copolyamide, partially aromatic homopolyamide, partially aromatic copolyamide, ter-polyamide, and mixtures thereof.

17. The thermoplastic film according to claim 2, wherein the polyamide is selected from the group consisting of: polyamide 6, polyamide 66, copolyamide 6/66, copolyamide 66/6, copolyamide 6/12, and terpolyamide 6/66/12.

18. The thermoplastic film according to claim 8, which has a water vapor transmission rate WVTR≥250 g/m²·day, at 23° C. and 90/5.

19. The film according to claim 8, which has an oxygen transmission rate analyzed in accordance with DIN 53 380 at 23° C. and 50% relative humidity using an Ox-Tran® permeation analyzer lower than 50 cc/m²·day.

20. Use of a packaging as defined in claim 11, to sterilize objects or materials by means of water vapor or ethylene oxide.
